(19) United States
(12) Patent Application Publication
(10) Pub. No.: US 2010/0317960 A1
(43) Pub. Date: Dec. 16, 2010

(54) THERMOTHERAPY DEVICE AND METHOD TO IMPLEMENT THERMOTHERAPY

(76) Inventors: Patrick Gross, Langensendelbach (DE); Karsten Hiltawsky, Schwerte (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/797,754

(22) Filed: Jun. 10, 2010

(30) Foreign Application Priority Data
Jun. 10, 2009 (DE) 10 2009 024 589.8

(51) Int. Cl. A61N 7/02; A61B 8/14; A61B 5/055

(52) U.S. Cl. 600/411; 601/3; 600/439

(57) ABSTRACT

A thermotherapy device has a transmitter, a receiver and a processing unit. The transmitter is designed to emit a high-energy radiation in a treatment region of a patient. The high-energy radiation exhibits a power that is suitable for thermotherapy. The receiver is designed to detect a sound signal that is generated by the treatment region depending on the high-energy radiation radiated into said treatment region. The processing unit is coupled with the transmitter) and the receiver. The processing unit automatically determines information about the treatment region depending on the detected sound signal.

### BACKGROUND OF THE INVENTION

1. Field of the Invention

The present invention concerns a thermotherapy device and a method to implement a thermotherapy, and in particular a device and method to control and monitor a therapeutically applied thermal energy deposition.

2. Description of the Prior Art

In a number of clinical fields, local, thermal energy deposition (application) is viewed as a promising therapeutic method. Present fields of application range from tumor ablation to the treatment of obesity or to thermally induced local pharmaceutical administration. Independent of the application, it is necessary that the administered energy be concentrated only in the desired body or tissue region and act only there, so healthy tissue and in particular critical structures (for example nerves) remain unaffected. A treatment implementation therefore includes the following functions:
define the target area and additional significant structures, for example endangered structures (for example nerves);
determine parameters for the therapy implementation, efficiency and effectiveness;
monitor the treatment process; and
determine the result.

According to the prior art, many modalities for diagnostic scanning or image generation—for example magnetic resonance imaging (MRI), positron emission tomography (PET), computed tomography (CT), ultrasound examination, etc.—can be used for the definition of the target area and additional significant structures. It is important for this information to be available in the coordinate system of the treatment devices and the treatment monitoring devices. This is frequently implemented by mutual registration or combination of diagnostic data sets in the treatment and monitoring reference framework. This is frequently implemented using multiple data sets obtained from multiple devices. In order to implement such a common registration, it is useful if the monitoring device able to either directly define the target area or additional structures or to define auxiliary orientation points which are visible both in the monitoring device and the diagnostic device. A magnetic resonance imaging or an ultrasound examination are advantageously used for a control of the therapy since they are able to provide many of the desired functions.

In order to plan and to monitor the thermal energy feed, a control device can additionally provide auxiliary information for what is known as a “treatment model”. This enables a determination of the parameters for the thermal energy feed. In the case of heat therapies, a perfusion which accompanies a cooling of the tissue is a very important parameter. In the prior art, for example, MRI or an ultrasound examination are used in order to define these parameters. For example, the treating physician can then use the vascularization in the region of the ablation in order to determine the required energy dose on the basis of his experiences. An additional important parameter is the oxygen enrichment in the blood. Heat therapies can be used in connection with additional therapies, for example a radiation therapy or, respectively, radiotherapy. In a radiotherapy it is desirable to increase the oxygen enrichment in the tissue in order to prevent the protective effect of a tissue hypoxia. In an additional combined therapy, chemotherapy is combined with thermotherapy. For example, special media have been developed which contain medicines that are stable in the bloodstream and are activated by thermal energy. For example, the thermal energy can be supplied by a focused ultrasound (8^{th} International Symposium on Therapeutic Ultrasound, “Creating the Infrastructure for Validated Ultrasound Guided Drug Delivery”, K. Ferrara, D. Kruse, et al., Minneapolis, USA, 10-13 Sep. 2008).

Although a few research prototypes and some products in the prior art supply thermal energy without the spatial and temporal temperature variation within the tissue actually having to be monitored, such approaches are very limited in their applicability. Examples of techniques which are presently used without temperature measurement are, for example, laser-induced thermotherapy (LITT, which is also known as laser ablation) and treatment with high-intensity focused ultrasound (HIFU, also known as FUS). An additional problem for such treatments if temperature is not monitored is in compliance with legal standards for using certain medical apparatuses, for example CE labeling and FDA requirements. The thermal energy supply and the reaction of the tissue to the applied energy are therefore typically monitored. Since magnetic resonance imaging (MRI) is able to measure a relative temperature, this technique has been used in order to monitor an ablation. However, the use of MRI has specific disadvantages: the device is very expensive, has a low temporal resolution, and the thermal treatment device can be used together with the magnetic resonance imaging only with difficulty.

An ultrasound examination is significantly less expensive, faster, and uses a device that is widespread among radiology specialists, for example urologists and gynecologists. An alternative approach is based on ultrasound, wherein temperature-dependent changes of a mechanical impedance are detected using ultrasound, as disclosed in U.S. Pat. No. 5,370,121. More recent developments in an imaging with shear waves using ultrasound are likewise promising, as described in U.S. Pat. Nos. 6,764,448 and 5,810,731. However, changes of the mechanical response occur only at a level at which irreversible tissue changes occur. This technique is therefore not applicable if only small temperature changes without tissue damage occur. However, this is desirable in the calibration for a local thermal energy deposition and for monitoring of tissue which should remain unaffected.

It has been shown that a direct measurement of the temperature is often difficult. Viktor I. Pasechnik suggests a method and proposes a simulation for a direct measurement of Brownian motion of the tissue using ultrasound (Publication 4aBB7, ASA/EAA/DAGA '99 Meeting, Berlin). However, it is unknown whether this method was ever realized. The noise differences which are generated by the temperature difference are most probably very small since an increase in body temperature by 10° K amounts to only approximately 3%.

Similar to the initial diagnostic imaging and treatment planning, there is a large set of devices in order to determine the result of the therapy. In order to determine the effectiveness and efficiency of the treatment, a common registration of the monitoring device in the treatment reference framework is advantageous.

### SUMMARY OF THE INVENTION

In light of the problem posed above in the prior art, it is therefore an object of the present invention to provide an improved device to implement a thermotherapy.

According to the present invention, a thermotherapy device is provided that comprises a transmitter, a receiver and a processing unit. The transmitter emits a high-energy radiation in a treatment region of a patient. The high-energy radiation exhibits a power that is suitable for thermotherapy. For example, the high-energy radiation can be in the form of one or more high-energy radiation pulses that exhibit a power suitable for thermotherapy and exhibit a length suitable for a thermoacoustic imaging. Such a pulse length amounts to one microsecond or less, for example. The receiver is in the position to detect a sound signal which is generated by the treatment region as a response to the high-energy radiation radiated into the treatment region. Such a radiation of a sound signal from the treatment region is also designated as thermoacoustic radiation from the treatment region is also designated as thermoacoustic radiation. Given an emission of the high-energy radiation in the form of high-energy radiation pulses, the sound signal can be detected with the receiver, synchronized to the emission of the high-energy radiation pulses. The processing device of the thermotherapy device is coupled with the transmitter and the receiver. The processing device is able to automatically determine information about the treatment region from the detected sound signal. For example, the information about the treatment region can comprise a temperature of the treatment region; a temperature change of the treatment region; an anatomical image of the treatment region; a structure of the treatment region; a structural change of the treatment region; a change of the physiology in the treatment region; a change of cellular markers in the treatment region; or a change of molecular markers in the treatment region.

Since the thermoacoustic effect provides information regarding locally emitted energy (depending on a coefficient of thermal expansion), the temperature of the treatment region can be determined therefrom.

It is thus possible with the thermotherapy device to monitor the treatment region during a thermotherapeutic treatment, for example with regard to a temperature increase or with the aid of a thermoacoustic imaging. An effect or a progress of the thermotherapy treatment in the treatment region can thereby be monitored during the treatment. No additional devices for monitoring of the treatment region are required, whereby a cost savings can occur and the thermotherapy can be implemented more quickly.

According to one embodiment, the processing unit is designed to emit additional high-energy radiation pulses with the aid of the transmitter, depending on the information determined about the treatment region. For example, if a desired target temperature in the treatment region was established at the beginning of the thermotherapy, the processing unit can continuously monitor the temperature in the treatment region via the treatment region and emit additional high-energy radiation pulses via the transmitter until the desired temperature is achieved in the treatment region. Alternatively, the information about the temperature in the treatment region or one of the aforementioned items of information about the treatment region can be provided to a treating physician who then allows the emission of additional high-energy radiation pulses depending on this information.

For example, the sound signal can be an ultrasound signal which can be detected with the aid of receivers that are known from thermoacoustic imaging. Moreover, the additional processing methods known from thermoacoustics can also be used, for example for imaging or to determine the temperature of the treatment region. Reliable methods are thereby available for monitoring the thermotherapy.

The thermotherapy device can also comprise an image processing device which is coupled with the processing unit. The image processing unit is in the position to generate image information of the treatment region from the information determined about the treatment region. The image processing device can also be designed such that it can determine a target structure of the treatment region and/or a significant structure in an environment of the treatment region or the target structure. With the aid of the image processing device it is thus possible to localize the target structure precisely and to monitor it during the thermotherapy. Moreover, it is possible to detect significant or critical structures (for example nerves) in order to avoid an energy deposition in these significant or critical structures.

The image processing device can also be designed so that it is in the position to determine a measure for the energy deposition in the treatment region. It is thereby possible to monitor an effectiveness of the thermotherapy, and possibly to align the thermotherapy more precisely on the target region via a modified arrangement or focusing of the transmitter.

The image processing device can also be designed such that it can determine a temperature in the treatment region, in particular in the target structure and in the significant or critical structure in the environment of the treatment region. The effectiveness of the thermotherapy during the treatment can thereby be monitored, and at the same time it can be ensured that adjoining critical regions that are not to be treated are actually not affected by the thermotherapy.

The high-energy radiation can comprise high-energy focused ultrasonic waves, radio-frequency waves or laser light waves. Since all three types of energy radiation lead to a heating of the target structure in the treatment region, all cited energy radiations are suitable for use of the thermotherapy device according to the invention.

According to a further embodiment, the transmitter of the thermotherapy device is also designed such that it can emit a low-energy radiation pulse in the treatment region given suitable activation. The low-energy radiation pulse is a radiation pulse with a power unsuitable for thermotherapy, i.e. it possesses a power which is too low for a thermotherapy, meaning that the radiation pulse is not strong enough to induce irreversible alterations in the treatment region. The receiver of the thermotherapy device is also designed such that it can detect sound signals which are generated by the treatment region depending on the low-energy radiation pulse radiated into the treatment region. The processing unit is in turn able to determine additional information about the treatment region from this sound signal. This additional information can, for example, be an anatomical image of the treatment region; a physiology in the treatment region; cellular markers in the treatment region; molecular markers in the treatment region; or a measure of the effectiveness of the low-energy radiation pulse in the treatment region. It is thereby possible to plan a thermotherapy with the thermotherapy device without already causing irreversible changes in the treatment region. Since both the low-energy radiation pulses for the examination of the treatment region and the high-energy radiation pulses for the actual treatment of the treatment region are generated with the same transmitter, it is automatically ensured that the transmitter possesses a suitable position to treat the target structure in the treatment region. Moreover, conclusions of an anticipated effectiveness of the high-energy radiation pulse in the treatment region can already be made in the planning of the thermotherapy with the aid of the low-energy radiation pulses.

The low-energy pulse can, for example, comprise ultrasonic waves which are suitable to generate ultrasound images of the treatment region. An optimal alignment of the transmitter with the treatment region can be set using the ultrasound images, and then the thermotherapy can be implemented with the aid of high-energy and focused ultrasonic waves with the same transmitter.

According to one embodiment, the device also comprises a magnetic resonance system which is designed to detect magnetic resonance information about the treatment region with the aid of a magnetic resonance measurement. The magnetic resonance information can, for example, be a temperature of the treatment region, a temperature change of the treatment region or an anatomical image of the treatment region. A precision of the planning and evaluation of the thermotherapy can be increased via the use of a combination of the thermotherapy device with the magnetic resonance system. The information about the treatment region that is detected with the aid of the receiver of the thermotherapy device before and during the thermotherapy can be compared with information of the magnetic resonance system detected before and during the thermotherapy. The information of the thermotherapy device is based on thermoacoustic effects, in contrast to which the information of the magnetic resonance system is based on magnetic resonance effects. Since thermoacoustic effects and magnetic resonance effects suffer from different interferences and distortions, such interferences and distortions can be compensated via a combination of the two types of information, and a higher precision can thus be achieved.

Furthermore, according to the present invention a method is provided to implement a thermotherapy. In the method a high-energy radiation pulse is sent into a treatment region of a patient. The high-energy radiation pulse has a power which is suitable for a thermotherapy, i.e. it has a power that produces an irreversible change in the treatment region. According to the method, a sound signal (advantageously an ultrasound signal) is detected which is generated by the treatment region as a response to the high-energy radiation pulse radiated into the treatment region. Information about the treatment region is determined automatically with the aid of the detected sound signal. For example, the information can be: a temperature of the treatment region; a temperature change of the treatment region; an anatomical image of the treatment region; a structure of the treatment region; a structural change of the treatment region; a change of the physiology in the treatment region; a variation of cellular markers in the treatment region; or a variation of molecular markers in the treatment region. The method thus enables information about the treatment region to be obtained during a treatment of the treatment region in order to control the treatment on the basis of this information, for example.

The high-energy radiation pulse can exhibit a length suitable for a thermoacoustic imaging. Such a pulse length can, for example, be one microsecond or less. The length of the high-energy radiation pulse determines the wavelength range of the sound waves of the sound signal which is generated by the treatment region depending on the high-energy radiation pulse radiated into the treatment region. For example, a pulse of 1 microsecond generates frequencies in a range from 0 to approximately 1 MHz. The higher the generated frequencies, the higher the resolution of a thermoacoustically generated image. Moreover, in thermoacoustic imaging the propagation speed of the sound signal in the biological tissue of the patient can also be used to generate the thermoacoustic image. For this purpose, the sound signal can be detected synchronized with the emission of the high-energy radiation pulse. Image information of the treatment region can then be generated with the aid of the detected sound signal. The generation of the image information can be generated with the aid of known methods for thermoacoustic imaging, for example.

Depending on the information generated from the sound signal—in particular image information of the treatment region or temperature information of the treatment region—additional high-energy radiation pulses can be emitted into the treatment region. A thermotherapy can thereby be controlled automatically or be terminated automatically upon exceeding predetermined limit values.

According to one embodiment, a target structure of the treatment region and/or a significant structure in an environment of the treatment region is determined with the aid of the automatically determined image information. It is thereby possible to adapt the target region during the thermotherapy in order to achieve a better therapy outcome on the one hand, and on the other hand to save significant or critical structures (for example nerves) from irreversible damage.

In a further embodiment of the method, an energy deposition in the treatment region is determined automatically with the aid of the previously determined image information. An effectiveness and efficiency of the thermotherapy in the treatment region can be monitored in that the energy deposition in the treatment region is determined.

The high-energy radiation pulse can be introduced into the treatment region with the aid of a transmitter as high-energy and focused ultrasonic waves, radio-frequency waves or laser light waves. The transmitter which emits the high-energy radiation pulse can, for example, be introduced into the patient in proximity to the treatment region with the aid of a probe or with the aid of a catheter, or can emit the high-energy radiation pulse into the examination region via the skin of the patient. In order to avoid an excessive thermal expansion in the treatment region between the transmitter and a tissue of the patient that is in contact with the transmitter, the transmitter can be provided with a corresponding cooling for the treatment region.

In an additional embodiment of the method, a low-energy radiation pulse is additionally emitted into the treatment region. The low-energy radiation pulse possesses a power that is unsuitable (i.e. too low) for thermotherapy. Sound signals which are generated by the treatment region depending on the low-energy radiation pulse radiated into the treatment region are detected, and information about the treatment region is determined automatically with the aid of the detected sound signals. The information can, for example, comprise an anatomical image of the treatment region; a physiology in the treatment region; cellular markers in the treatment region; molecular markers in the treatment region; or a measure of the efficiency of the low-energy radiation pulse in the treatment region.

The low-energy radiation pulse can moreover comprise ultrasonic waves which are suitable to generate ultrasound images of the treatment region. The sound signals generated by the treatment region as a response to the radiated low-energy radiation pulse in this case comprise ultrasonic waves which are detected and can be used to generate an ultrasound image of the treatment region. For example, with the use of the ultrasound image it is possible to identify a determination of a target structure in the treatment region and to determine in advance significant or critical structures which should be excepted from an exposure with a high-energy radiation pulse, without having to emit high-energy radiation pulses into the treatment region. A very precise planning of the subsequent thermotherapy can thus be implemented in a closed method.

According to a further embodiment, magnetic resonance information of the treatment region is additionally determined with the aid of a magnetic resonance measurement. The magnetic resonance information can, for example, comprise a temperature of the treatment region, a temperature change of the treatment region or an anatomical image of the treatment region. Since a magnetic resonance measurement suffers from different interferences and distortions than an ultrasound measurement, a more precise and reliable planning and implementation of the thermotherapy can be ensured via the combination of the ultrasound measurement with the magnetic resonance measurement.

### BRIEF DESCRIPTION OF THE DRAWINGS

FIG. 1 schematically illustrates a thermotherapy device according to an embodiment of the present invention.

FIG. 2 is a flowchart of an embodiment of a method to implement a thermotherapy according to the present invention.

### DESCRIPTION OF THE PREFERRED EMBODIMENTS

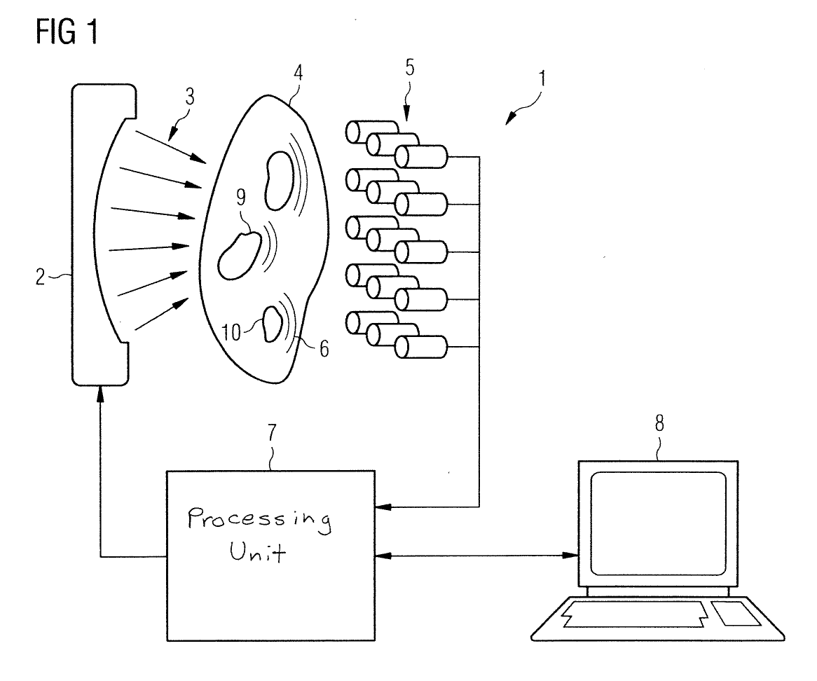

In FIG. 1 a device 1 is shown which is suitable for implementation of a thermotherapy. The device 1 comprises a transmitter 2, a receiver arrangement 5, a processing unit 7 and an image processing device 8. The transmitter 2 can be, for example, a transmitter to emit a high-energy radiation which, for example, emits high-energy focused ultrasonic waves, radio-frequency waves or laser light waves. The transmitter 2 is designed such that it can emit an energy radiation with such a high energy that the energy is suitable to produce irreversible changes in a treatment region 4 of a patient. For example, the power that can be emitted by the transmitter 2 is so high that it is suitable for a tumor ablation in the treatment region 4. for example, the transmitter 2 can be introduced into proximity of the treatment region 4 in the patient with the aid of a probe or a catheter. In order to avoid an overheating of a tissue region which is in direct contact with an energy radiation surface of the transmitter 2, this energy radiation surface of the transmitter can possess a cooling system. If the transmitter is an ultrasound transmitter, the transmitter can be designed with a relatively large transmission surface and a correspondingly large focusing lens in order to keep the energy density at the output of the transmitter low in order to avoid a heating of adjoining tissue. In particular given a use of a laser as a transmitter, a cooling of the laser tip can be necessary.

Among other things, two different tissue types 9 an 10 are located in the treatment region 4 shown in FIG. 1. For example, the tissue type 9 represents a tumor tissue which should be damaged and destroyed with the use of thermotherapy. In contrast to this, the tissue 10 represents an additional significant structure in the treatment region 4 which should not be damaged by the thermotherapy (since it is nerve tissue, for example). As is shown in FIG. 1, the transmitter 2 is aligned toward the treatment region 4 of the patient such that the energy radiation 3 emitted by the transmitter 2 specifically affects the tissue type 9 in the treatment region 4.

In addition to the emission of the high-energy radiation, the transmitter 2 is also suitable for emission of a low-energy radiation that has such a low energy that it produces no irreversible changes in the treatment region 4. However, both the high-energy radiation and the low-energy radiation that can be emitted by the transmitter 2 are suitable to produce thermoacoustic reactions of the tissue 9, 10 in the examination region 4. Both the high-energy radiation and the low-energy radiation are emitted into the examination region 4 by the transmitter 2 only in short pulses, for example, of a length of 1 microsecond or shorter. These short pulses produce a heating of the different tissue 9, 10 in the examination region 4. Due to the heating a thermal expansion occurs that leads to the emission of ultrasonic waves 6. The different tissues 9, 10 generate ultrasonic waves with different intensity and different spectrum. The generated ultrasonic waves 6 are detected by the receiver arrangement 5. A matrix-like arrangement of multiple individual receivers is shown in FIG. 1, such that a thermoacoustic imaging can be implemented with the aid of a suitable processing of the acoustic signals received at the individual receivers, as is known in the prior art. The processing unit 7 controls the transmitter 2 accordingly and receives the detected ultrasound signals from the receivers synchronized with the activation of the transmitter 2. An image of the treatment region 4 is generated from the received ultrasound signals with the aid of the image processing device 8.

It is thus possible with the thermotherapy device 1 shown in FIG. 1 to plan a thermotherapy—i.e. to precisely examine the treatment region 4, to suitably align the transmitter 2 accordingly in order to protect significant tissue regions 10 to be excepted from the thermotherapy, and simultaneously to bring the tissue regions 9 that are to be treated exactly into the focus of the transmitter 2—and to continuously monitor the treatment outcome during the treatment with the high-energy radiation.

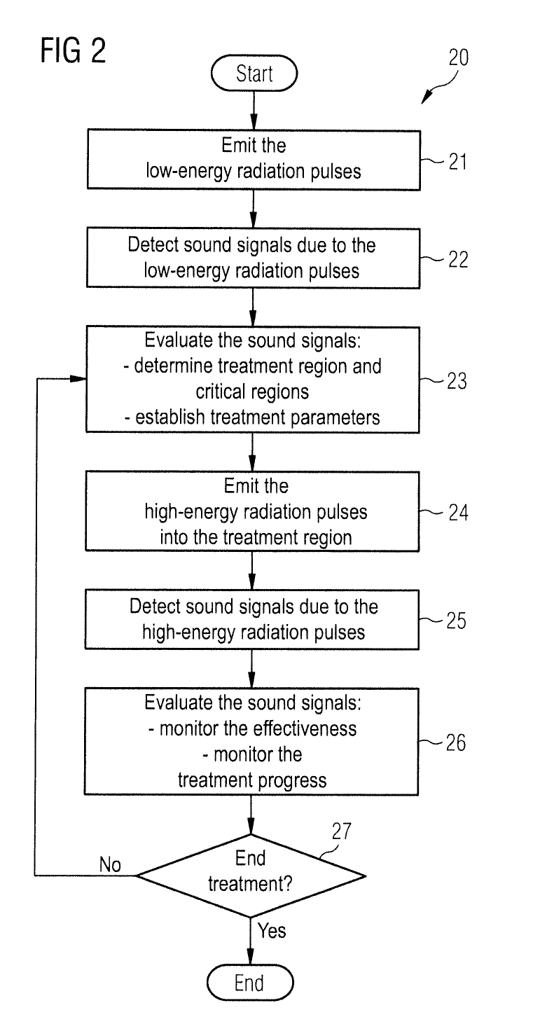

In connection with FIG. 2, the workflow of a thermotherapy with the aid of the thermotherapy device 1 shown in FIG. 1 is subsequently described in detail using a workflow diagram 20.

In the thermotherapy device 1 of FIG. 1, the thermoacoustic effect is used in order to plan and monitor a therapeutic energy deposition. First the target structure for a thermotherapy and additional significant structures (for example nerves or other vulnerable structures) are defined in that an anatomy, a structure, a physiology (for example a perfusion or cellular or molecular markers) are determined, for example in connection with contrast agents. As is shown in Block 21 in the diagram 20, for this low-energy radiation pulses are emitted (Block 21) which produce a thermal expansion of the tissue 9, 10 in the treatment region 4 and thereby generate ultrasonic waves 6. In Block 22 the ultrasonic signals that are generated in this way are detected by the receiver arrangement 5 and processed into thermoacoustic images with the aid of the processing unit 7 and the image processing device 8.

Parameters which are relevant to the process of the therapeutic energy deposition are then determined on the basis of this image information in order, for example, to determine an efficiency or effectiveness of the energy deposition under consideration of (for example) a perfusion of the tissue which involves a cooling of the tissue. Moreover, the critical regions (for example nerve structures) are identified and the parameters of the therapeutic energy deposition are accordingly adapted (Block 23).

High-energy radiation pulses are then emitted into the treatment region 4 (Block 24). The ultrasonic signals produced by the tissue expansion are detected synchronized with the emission of the high-energy radiation pulses (Block 25). Via evaluation of the sound signals, the effectiveness of the energy deposition can be directly monitored and a treatment progress can be determined, for example in that a tissue expansion is measured or a change in the anatomy or the physiology or of cellular/molecular markers is monitored (Block 25). In block 27 it is determined (using a treatment progress which was determined in Block 26) whether the treatment should be continued or ended. This can either be determined automatically by (for example) the processing unit 7 from predetermined parameters or be decided via a corresponding dialog with a treating physician via a user interface of the image processing device 8. If the treatment is continued, this can be continued in Block 23, for example, so that the sound signals produced by the high-energy radiation pulses are possibly used in order to readjust treatment parameters. Alternatively, the treatment can also be directly continued with unchanged treatment parameters in Block 24.

Finally, the result of the treatment is determined by determining an anatomy or structure, a physiology (for example a perfusion) or cellular or molecular markers (for example in connection with contrast agents) in that—as described above—low-energy radiation pulses are emitted by the transmitter 2 and corresponding ultrasonic signals due to the low-energy radiation pulses are detected by the receiver arrangement 5 and are evaluated for a thermoacoustic imaging with the aid of the processing unit 7 and the image processing device 8.

The described method can be combined in a simple manner with conventional ultrasound examinations (for example A-mode, B-mode, M-mode, Doppler mode, shear wave ultrasound etc.). Moreover, the method can be combined with a magnetic resonance imaging, whereby an increased accuracy can be achieved in the planning and evaluation since ultrasound and magnetic resonance suffer from different interferences and distortions which can be mutually compensated given a combination of the two methods.

The device according to the invention and the method according to the invention can be used in all required steps of a thermotherapy:
Definition of the target structure and additional significant structures

Since a thermoacoustic diagnosis is enabled with the aid of the device according to the invention and the method according to the invention, a wider range of possible target areas and diagnostic examinations is covered. For example, a photoacoustic imaging (as one category of thermoacoustic imaging) can generate images for specific optical frequencies which provide the basis of an imaging of a blood oxygen enrichment or for fluorescence markers. A microwave-based thermoacoustic imaging can be used in order to show the electromagnetic impedance of tissue. Therefore the thermoacoustic imaging is suitable to directly depict the target pathology and additional significant structures. A common processing with additional imaging devices is in particular possible in combination with an ultrasound imaging.
Parameter determination

Thermoacoustic imaging—in particular in combination with an ultrasound imaging—offers the possibility to directly assess parameters which are relevant to the planning and application of thermotherapies. This includes an assessment of a perfusion, an oxygen enrichment, an accumulation of chemical substances and drug carriers.
Monitoring of the thermal energy feed

The thermoacoustic effect—including the optoacoustic effect—enables local temperature increases to be shown using ultrasound. This is achieved in that pressure waves are detected that are caused by the local thermal expansion. If the radiation is supplied in short pulses that are sufficiently short in order to avoid a diffusion, the signal which is emitted by a thermally excited volume can be considered as a spherical ultrasound source which depends on the absorbed energy and the coefficients of thermal expansion. The positions of the sources an their intensities can be reconstructed via measurement of the emitted ultrasonic signal from multiple direction, as this is possible with the aid of the receiver arrangement 5 shown in FIG. 1, for example. The target area can thus be monitored in a simple manner during the thermotherapy.
Determination of the result

Since it is possible with the aid of a thermoacoustic imaging to assess anatomical, physiological and cellular/molecular markers, the result of a thermotherapy can be directly determined from this. This can in particular be implemented with the use of thermoacoustic imaging.

Although modifications and changes may be suggested by those skilled in the art, it is the intention of the inventors to embody within the patent warranted hereon all changes and modifications as reasonably and properly come within the scope of their contribution to the art.

We claim as our invention:

1. A thermotherapy device comprising:
a radiation transmitter that emits high-energy radiation into a treatment region of a patient, said high-energy radiation having a thermotherapy-implementing power;
a receiver that detects a sound signal generated by said treatment region dependent on the high-energy radiation radiated into the treatment region, said receiver emitting a receiver output corresponding to the detected sound signal; and
a processing unit connected to said transmitter and said receiver that automatically determines information characterizing said treatment region from said receiver output signal.

2. A device as claimed in claim 1 wherein said processing unit is configured to control said transmitter to emit said high-energy radiation as a high-energy radiation pulse having a length for thermoacoustic imaging, and to operate said receiver to detect said sound signal synchronized with emission of said high-energy radiation pulse.

3. A device as claimed in claim 2 wherein said processing unit is configured to control said transmitter to emit additional high-energy radiation pulses dependent on said information.

4. A device as claimed in claim 1 wherein said receiver detects an ultrasound signal as said sound signal.

5. A device as claimed in claim 1 comprising an image processor connected to said processing unit, said image processor being configured to generate image information characterizing said treatment region from said information characterizing said treatment region generated by said processing unit.

6. A device as claimed in claim 5 wherein said image processor is configured to determine at least one of a target structure of said treatment region and a prominent structure in an environment of said treatment region.

7. A device as claimed in claim 5 wherein said image processor is configured to determine an energy deposition in said treatment region.

8. A device as claimed in claim 5 wherein said image processor is configured to determine a temperature in said treatment region.

9. A device as claimed in claim 1 wherein said transmitter is a transmitter is a transmitter selected from the group consisting of high-energy focused ultrasound wave transmitters, radio-frequency wave transmitters, and laser light wave transmitters.

10. A device as claimed in claim 1 wherein said processing unit is configured to determine information characterizing said treatment region selected from the group consisting of a temperature of the treatment region, a temperature change of the treatment region, an anatomical image of the treatment region, a structure of the treatment region, a structural change of the treatment region, a change in physiology of the treatment region, a change in cellular markers in the treatment region, and a change in molecular markers in the treatment region.

11. A device as claimed in claim 1 wherein said transmitter is configured to also emit a low-energy radiation pulse into said treatment region, said low-energy radiation pulse having a power that is not capable of implementing thermotherapy, and wherein said receiver is configured to detect an additional sound signal generated in said treatment region dependent on said low-energy radiation pulse radiated into the treatment region, and to emit an additional receiver output signal corresponding to the detected additional sound signal, and wherein said processing unit is configured to determine additional information characterizing the treatment region dependent on said additional output signal from said receiver.

12. A device as claimed in claim 11 wherein said processing unit is configured to determine said additional information from the group consisting of an anatomical image of the treatment region, physiology in the treatment region, cellular markers in the treatment region, molecular markers in the treatment region, and a measure of effectiveness of said low-energy radiation pulse in said treatment region.

13. A device as claimed in claim 11 wherein said transmitter is configured to emit ultrasonic waves as said low-energy pulse, said ultrasonic waves having a power to generate ultrasound images of the treatment region.

14. A device as claimed in claim 1 comprising a magnetic resonance system that acquires magnetic resonance data characterizing the treatment region in a magnetic resonance data acquisition procedure.

15. A device as claimed in claim 14 wherein said magnetic resonance system is configured to detect magnetic resonance information selected from the group consisting of a temperature of the treatment region, a temperature change of the treatment region and an anatomical change of the treatment region.

16. A thermotherapy method comprising the steps of:
emitting high-energy radiation into a treatment region of a patient, said high-energy radiation having a thermotherapy-implementing power;
detecting a sound signal generated by said treatment region dependent on the high-energy radiation radiated into the treatment region; and
in a computerized processor automatically determining information characterizing said treatment region from the detected sound.

17. A method as claimed in claim 16 comprising, from said processor controlling a transmitter to emit said high-energy radiation as a high-energy radiation pulse having a length for thermoacoustic imaging, and operating a receiver to detect said sound signal synchronized with emission of said high-energy radiation pulse.

18. A method as claimed in claim 17 comprising controlling said transmitter to emit additional high-energy radiation pulses dependent on said information.

19. A method as claimed in claim 16 comprising detecting an ultrasound signal as said sound signal.

20. A method as claimed in claim 16 comprising, in an image processor connected to said processor, generating image information characterizing said treatment region from said information characterizing said treatment region generated by said processing unit.

21. A method as claimed in claim 20 comprising in said image processor, determining at least one of a target structure of said treatment region and a prominent structure in an environment of said treatment region.

22. A method as claimed in claim 20 comprising, in said image processor, determining an energy deposition in said treatment region.

23. A method as claimed in claim 20 comprising in said image processor determining a temperature in said treatment region.

24. A method as claimed in claim 16 comprising emitting said high-energy radiation selected from the group consisting of high-energy focused ultrasound, radio-frequency waves, and laser light.

25. A method as claimed in claim 16 comprising in said processor determining information characterizing said treatment region selected from the group consisting of a temperature of the treatment region, a temperature change of the treatment region, an anatomical image of the treatment region, a structure of the treatment region, a structural change of the treatment region, a change in physiology of the treatment region, a change in cellular markers in the treatment region, and a change in molecular markers in the treatment region.

26. A method as claimed in claim 16 comprising to also emitting a low-energy radiation pulse into said treatment region, said low-energy radiation pulse having a power that is not capable of implementing thermotherapy, and detecting an additional sound signal generated in said treatment region dependent on said low-energy radiation pulse radiated into the treatment region, and in said processor determining additional information characterizing the treatment region dependent on the detected additional sound signal.

27. A method as claimed in claim 26 comprising in said processor determining said additional information from the group consisting of an anatomical image of the treatment region, physiology in the treatment region, cellular markers in the treatment region, molecular markers in the treatment region, and a measure of effectiveness of said low-energy radiation pulse in said treatment region.

28. A method as claimed in claim 26 comprising emitting ultrasonic waves as said low-energy pulse, said ultrasonic waves having a power to generate ultrasound images of the treatment region.

29. A method as claimed in claim 16 comprising acquiring magnetic resonance data characterizing the treatment region in a magnetic resonance data acquisition procedure.

30. A method as claimed in claim 29 comprising, in said magnetic resonance data acquisition procedure, detecting magnetic resonance information selected from the group consisting of a temperature of the treatment region, a temperature change of the treatment region and an anatomical change of the treatment region.
